(19) United States
(12) Patent Application Publication
(10) Pub. No.: US 2023/0139236 A1
(43) Pub. Date: May 4, 2023

(54) NONINVASIVE ELECTRICAL STIMULATION METHOD AND DEVICE

(71) Applicant: Chang-dong LEE, Wonju-si (KR)

(72) Inventor: Chang-dong LEE, Wonju-si (KR)

(21) Appl. No.: 17/645,297

(22) Filed: Dec. 20, 2021

(30) Foreign Application Priority Data
Oct. 29, 2021 (KR) 10-2021-0146217
Nov. 25, 2021 (KR) 10-2021-0164543

(51) Int. Cl. A61N 1/36; A61N 1/04

(52) U.S. Cl. CPC A61N 1/36034; A61N 1/0404

(57) ABSTRACT

The present invention relates to a noninvasive electrical stimulation method and device, and the noninvasive electrical stimulation method includes the steps of: arranging first and second electrodes for applying electrical stimulation to a human body and first and second magnetic materials to thus allow the first and second magnetic materials to generate a magnetic field in a direction crossing the direction of an electric current between the first and second electrodes; and controlling at least one of a crossing angle (θ) at which the electric current between the first and second electrodes and the magnetic field generated by the first and second magnetic materials cross each other and the strength (B) of the magnetic field generated by the first and second magnetic materials.

### CROSS-REFERENCE TO RELATED APPLICATIONS

This application claims priority to and the benefit of Korean Patent Application No. 10-2021-0146217 filed with the Korean Intellectual Property Office on Oct. 29, 2021 and Korean Patent Application No. 10-2021-0164543 filed with the Korean Intellectual Property Office on Nov. 25, 2021, the disclosures of which are incorporated herein by reference in their entirety.

### TECHNICAL FIELD

The present invention relates to a noninvasive electrical stimulation method and device that is capable of attaching electrodes to specific regions of a human body to apply noninvasive electrical stimulation to the specific regions.

### BACKGROUND

Methods for stimulating cranial nerves to obtain specific treatment effectiveness include deep electrical stimulation, transcranial magnetic stimulation (TMS), transcranial electrical stimulation (TES), transcranial direct current stimulation (tDCS), transcranial random noise stimulation (tRNS), and the like.

Further, deep brain stimulation (DBS) is a surgical/invasive treatment technique that implants a medical device called a brain pacemaker within specific regions of the brain, and the brain pacemaker measures signals of the brain and applies electrical stimulation to specific regions of the brain.

The DBS is effective in treating chronic pain, Parkinson's disease, scrapie, dystonia, and the like, but has potentially serious side effects and medical complications.

Further, examples of noninvasive treatment techniques include noninvasive vagus nerve stimulation (nVNS), external trigeminal nerve stimulation (e-TNS), TMS, tDCS, and the like.

The nVNS noninvasively stimulates vagus nerves connected to central nerves from the outside to help autonomic functions and a brain's neural network activated, and in specific, the nVNS stimulates the vagus nerves under the ears or neck to indirectly stimulate the brain, thereby having influences on the functions of serotonin, norepinephrine, and the like related to mood stabilization.

The e-TNS stimulates trigeminal nerves on the external surface of the face such as the forehead to control the brain region related to mood stabilization, which is helpful in treating migraine and depression.

The TMS is carried out to allow a magnetic coil over the surface of the head to apply a very short magnetic field to the head at a shorter period of time than one millisecond (ms), so that the magnetic field with a strength of about 1 to 2 Tesla passes through the skull to induce the flow of an electric current in a very short time.

Further, the tDCS transmits weak direct current stimulation to the surface of the brain through electrodes positioned on the skin of the head to produce voluntary activation of neuron cells, thereby normalizing the functions of the brain and releasing symptoms. If drug treatments are not effective or if it is hard to apply the drug treatments due to the side effects of drugs, the tDCS may be helpful.

The treatment methods using the electrical stimulation as mentioned above have been applied to various fields over a long time, but as the human body is constituted of various substances, permittivity is differently distributed according to the body regions, so that it is hard to apply electrical stimulation to a desired position. Accordingly, generally, the electrical stimulation is applied to a treatment in which stimulation on a specific target region is not needed, for example, a muscle contraction treatment, and otherwise, the electrical stimulation is invasively applied by directly inserting electrodes into positions where stimulation is needed. Further, the electrical stimulation is applied by controlling electrodes in position to allow an electric current path to pass through positions to be stimulated.

The noninvasive cranial nerve stimulation such as the tDCS has advantages of low treatment costs and many conveniences of patients when compared to the invasive methods, but has to calculate the electric current path accurately and undesirably applies unnecessary stimulation to regions not desired. Besides, it is not easy that the electrodes come into close contact with the skin of a region with hairs, and accordingly, a danger of burns on the skin of the region with hairs may exist due to the electrical energy intensively collected thereto.

### SUMMARY

Accordingly, the present invention has been made in view of the above-mentioned problems occurring in the related art, and it is an object of the present invention to provide a noninvasive electrical stimulation method and device that is capable of easily stimulating a desired region of a human body.

To accomplish the above-mentioned objects, according to one aspect of the present invention, there is provided a noninvasive electrical stimulation method including the steps of: arranging first and second electrodes for applying electrical stimulation to a human body and first and second magnetic materials to thus allow the first and second magnetic materials to generate a magnetic field in a direction crossing the direction of an electric current between the first and second electrodes; and controlling at least one of a crossing angle (θ) at which the electric current between the first and second electrodes and the magnetic field generated by the first and second magnetic materials cross each other and the strength (B) of the magnetic field generated by the first and second magnetic materials.

In this case, the first and second magnetic materials are permanent magnets or electromagnets with different polarities from each other.

To accomplish the above-mentioned objects, according to another aspect of the present invention, there is provided a noninvasive electrical stimulation device including: an electrode part having first and second electrodes adapted to apply electrical stimulation; a magnetic force generation part having first and second magnetic materials adapted to generate a magnetic field in a direction crossing the direction of an electric current between the first and second electrodes; and a controller for controlling at least one of a crossing angle (θ) at which the electric current between the first and second electrodes and the magnetic field generated by the first and second magnetic materials cross each other and the strength (B) of the magnetic field generated by the first and second magnetic materials.

Further, at least some steps of the noninvasive electrical stimulation method according to the present invention may be implemented through a computer-readable storage medium for reading a program executable on a computer through the computer, and otherwise, they may be provided as a program itself.

According to the present invention, the plurality of electrodes and the plurality of magnetic materials are arranged to allow the electric current of the electrodes and the magnetic field generated by the magnetic materials to cross each other, and as the crossing angle or the strength of the magnetic field is controlled, accordingly, the electric current path is varied in depth, thereby easily stimulating the target regions, without changing the electrodes for electrical stimulation in position.

According to the present invention, the electrodes, which are disposed on the inner surface of the curved-shaped electrode part, come into contact with the forehead region of the human body to stably apply the electrical stimulation to the brain, and further, the electric current path between the electrodes is controlled in depth using the magnetic materials arranged on the inner surface of the electrode part, together with the electrodes, so that even if the electrodes come into contact with the forehead region, the electrical stimulation can be applied to a target region of the brain with various depths.

### BRIEF DESCRIPTION OF THE DRAWINGS

FIG. 1 is a block diagram showing an overall configuration of a noninvasive electrical stimulation device according to the present invention.

FIG. 2 is an exemplary view showing arrangements of electrodes and magnetic materials of the noninvasive electrical stimulation device according to the present invention.

FIG. 3 is an exemplary view showing an electric current path between the electrodes of the noninvasive electrical stimulation device according to the present invention.

FIG. 4 is a flowchart showing a noninvasive electrical stimulation method according to the present invention.

FIG. 5 is exemplary views showing methods for controlling an angle at which an electric current and a magnetic field cross each other according to the present invention.

FIG. 6 is exemplary views showing methods for changing an electric current path between electrodes in depth according to the present invention.

FIG. 7 is exemplary views showing methods for controlling a strength of the magnetic field according to the present invention.

FIG. 8 is a block diagram showing an overall configuration of a noninvasive electrical stimulation system according to the present invention.

FIGS. 9 to 11 are exemplary views showing another example of the noninvasive electrical stimulation device according to the present invention.

### DETAILED DESCRIPTION

Hereinafter, an explanation of a noninvasive electrical stimulation method and device according to the present invention will be given in detail with reference to the attached drawings.

In the description, if it is determined that the detailed explanation on the well known technology related to the present invention makes the scope of the present invention not clear, the explanation will be avoided for the brevity of the description. Further, the terms as will be discussed later are defined in accordance with the functions of the present invention, but may be varied under the intention or regulation of a user or operator. Therefore, they should be defined on the basis of the whole scope of the present invention.

Further, the present invention avoids explanations of parts already provided for respective systems or removes the functional configuration of the system typically provided in the art, so as to efficiently explain the technological parts of the invention, and accordingly, the functional configuration, which has to be additionally provided for the invention, will be explained hereinafter.

It is to be appreciated that those skilled in the art can easily understand the functions of the parts already used in conventional practices among the functional configurations not shown below and obviously understand the relations between the parts not explained and the parts added in the invention.

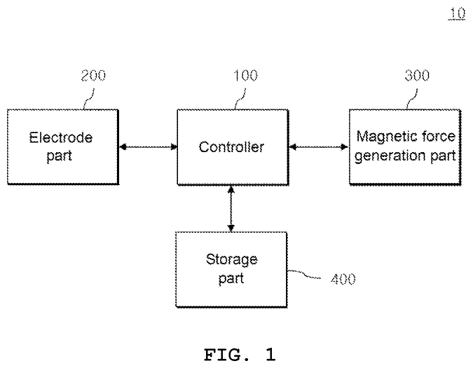

FIG. 1 is a block diagram showing an overall configuration of a noninvasive electrical stimulation device according to the present invention, and as shown, a noninvasive electrical stimulation device 10 according to the present invention may include a controller 100, an electrode part 200, a magnetic force generation part 300, and a storage part 400.

According to the present invention, a tDCS device is used as the noninvasive electrical stimulation device 10, and without being limited thereto, various devices, which are capable of applying noninvasive electrical stimulation using electrodes coming into contact with a human body, such as an nVNS device, a tRNS device, and the like, may be used as the noninvasive electrical stimulation device 10.

For example, the electrical stimulation device 10 may become a device configured to attach electrodes to a user's head or to be worn on his or her head to thus apply electrical stimulation to his or her brain, and otherwise, the electrical stimulation device 10 may be configured to allow an electric current to flow to a specific region of the head to thus apply electrical stimulation to a target region of the brain.

Hereinafter, for example, an explanation of the electrical stimulation device 10 configured to apply electrical stimulation to the brain will be given, but without being limited thereto, of course, the electrical stimulation device 10 according to the present invention may be applied even in the case where electrical stimulation may be applied to other specific regions except the brain.

Referring to FIG. 1, the controller 100 controls overall operations of the electrical stimulation device 10 and starting and finishing operations of the electrode part 200.

The electrode part 200 serves to apply electrical stimulation to a user's body and includes first and second electrodes attached to a specific region of the body.

For example, the first and second electrodes are anode and cathode, and as an electric current flowing between the first and second electrodes forms an electric current path on the surface of the brain to a given depth, direct current stimulation is applied to specific regions of the brain.

Further, the magnetic force generation part 300 serves to generate a magnetic field in a direction crossing the direction of the electric current between the first and second electrodes and includes first and second magnetic materials.

The first and second magnetic materials are arranged close to the first and second electrodes and have an influence on the electric current path between the first and second electrodes to allow a target region to which electrical stimulation is applied to be controlled through the change in the electric current path.

For example, the first and second magnetic materials are permanent magnets or electromagnets with different polarities from each other.

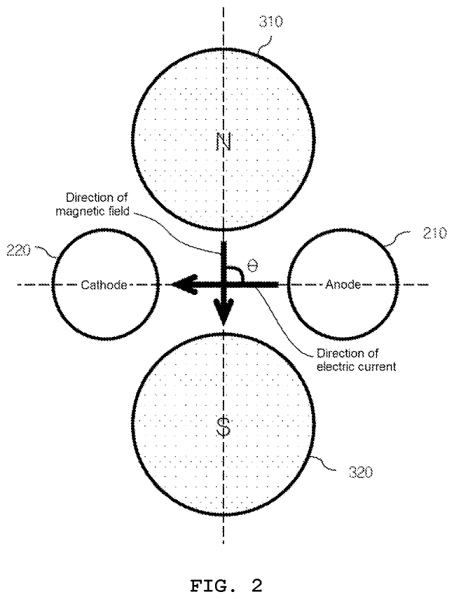

As shown in FIG. 2, for example, the anode 210 and the cathode 220 as the first electrode and the second electrode and the magnet 310 with a north pole and the magnet 320 with a south pole as the first magnetic material and the second magnetic material are arranged to thus allow a first line connecting the centers of the anode 210 and the cathode 220 and a second line connecting the centers of the magnet 310 with the north pole and the magnet 320 with the south pole to cross each other at a given angle θ.

Accordingly, the direction of the electric current between the anode 210 and the cathode 220 and the direction of the magnetic field generated by the magnet 310 with the north pole and the magnet 320 with the south pole cross each other at the given angle θ.

If the direction of the electric current between the first and second electrodes and the direction of the magnetic field generated by the first and second magnetic materials cross each other, as mentioned above, the strength of a magnetic field applied to the electric current path is obtained by the following Table 1:

F=qυB(sinθ)=iL×B(sinθ) [Table 1]

wherein L indicates the size of an electric current, B the strength of a magnetic field, and θ an angle at which the direction of the electric current and the direction of the magnetic field cross each other.

That is, the strength of the magnetic force applied to the electric current path between the first and second electrodes through the magnetic field generated by the first and second magnetic materials may be changed by the crossing angle θ and the strength B of the magnetic field.

In specific, when the crossing angle θ between the electric current and the magnetic field is 90°, the strength of the magnetic force is greatest, and as the crossing angle θ is close to 0 or 180°, the strength of the magnetic force becomes weakened.

As the strength B of the magnetic field generated by the first and second magnetic materials increases, further, the strength of the magnetic force applied to the electric current path becomes high.

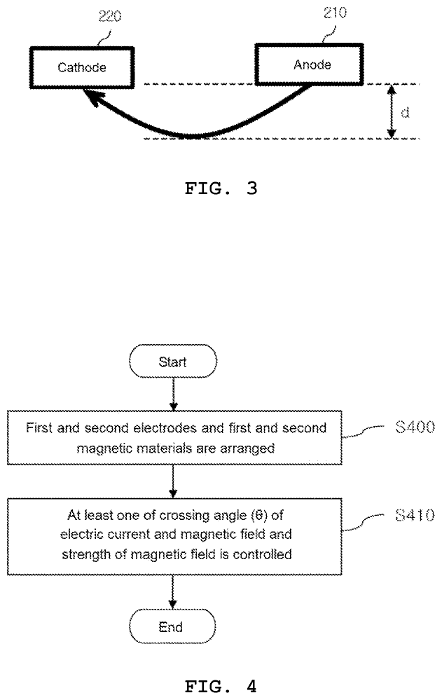

Referring to FIG. 3, the electric current path between the anode 210 and the cathode 220 has a given depth d to apply electrical stimulation from the surface to which they are attached to a specific region of an interior of the brain located on the electric current path.

As mentioned above, if the strength of the magnetic force applied to the electric current path between the anode 210 and cathode 220 is changed by the magnetic materials 310 and 320, the depth d of the electric current path between the anode 210 and cathode 220 may be controlled.

Accordingly, the controller 100 controls the angle θ at which the electric current between the first and second electrodes and the magnetic field generated by the first and second magnetic materials cross each other, or the strength B of the magnetic field generated by the first and second magnetic materials, so that the depth d of the electric current path between the first and second electrodes is controlled.

If the depth d of the electric current path between the first and second electrodes is controlled, like this, the target region in the interior of the brain, to which electrical stimulation is applied, may be varied, and accordingly, the electric current path may be formed more deeply in the surface of skin, thereby allowing the electrical stimulation to be applied to the deep portion of the brain or preventing the electrical stimulation from being applied to unnecessary regions where no electrical stimulation is needed.

Further, the storage part 400 serves to store software for the above-mentioned operations of the controller 100 or a protocol for electrical stimulation.

According to the present invention, as mentioned above, the plurality of electrodes and the plurality of magnetic materials are arranged to allow the electric current of the electrodes and the magnetic field generated by the magnetic materials to cross each other, and as the crossing angle or the strength of the magnetic field is controlled, accordingly, the electric current path is varied in depth, thereby easily stimulating the target region, without changing the positions of the electrodes for electrical stimulation.

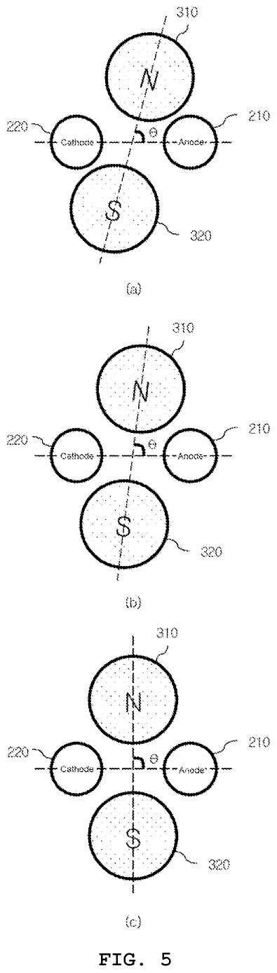

Now, an explanation of a noninvasive electrical stimulation method according to the present invention will be given in detail with reference to FIGS. 4 to 7c.

FIG. 4 is a flowchart showing a noninvasive electrical stimulation method according to the present invention, and the repeated explanation on the same parts as mentioned above with reference to FIGS. 1 to 3 will be avoided.

Referring to FIG. 4, a noninvasive electrical stimulation method according to the present invention includes the step of arranging first and second electrodes for applying electrical stimulation and first and second magnetic materials to thus allow the first and second magnetic materials to generate a magnetic field in a direction crossing the direction of an electric current between the first and second electrodes (at step S400).

Next, the noninvasive electrical stimulation method according to the present invention includes the step of controlling at least one of a crossing angle θ at which the electric current between the first and second electrodes and the magnetic field generated by the first and second magnetic materials cross each other, and the strength B of the magnetic field generated by the first and second magnetic materials (at step S410).

For example, the step (S410) of controlling the crossing angle θ or the strength B of the magnetic field is carried out at a time point when the electrical stimulation device 10 starts to operate, at a time point when the target region of the brain to which electrical stimulation is applied is changed, or in a process of calibrating the electrical stimulation device 10. However, the present invention is not limited thereto.

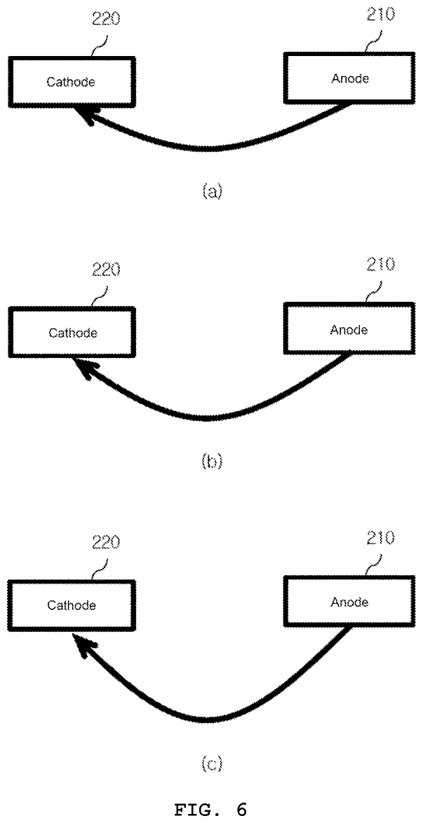

FIGS. 5a to 5c are exemplary views showing methods for controlling an angle θ at which the electric current and the magnetic field cross each other according to the present invention.

Referring to FIGS. 5a to 5c, the crossing angle θ between the electric current between the first and second electrodes and the magnetic field generated by the first and second magnetic materials increases in the order of FIG. 5a, FIG. 5b, and FIG. 5c, and in FIG. 5c, the crossing angle θ is 90°. As the crossing angle θ between the electric current and the magnetic field increases to come close to 90°, like this, the electric current path between the electrodes 210 and 220 may become deep in depth.

For example, if the permanent magnets with north and south poles are used as the first and second magnetic materials 310 and 320, the first and second magnetic materials 310 and 320 move in position in a state where the attached positions of the first and second electrodes 210 and 220 are fixed, and accordingly, an angle θ between a first line connecting the centers of the first and second electrodes 210 and 220 and a second line connecting the centers of the first and second magnetic materials 310 and 320 is changed. As the crossing angle θ comes close to 90°, in this case, the electric current path between the electrodes 210 and 220 may become deep in depth.

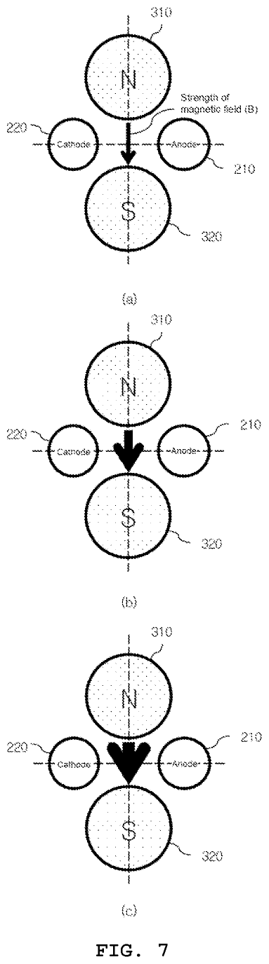

FIGS. 6a to 6c are exemplary views showing the corresponding electric current paths to the crossing angles θ as shown in FIGS. 5a to 5c, and as the crossing angle θ increases to come close to 90°, in this case, the electric current path may become deep in depth.

On the other hand, even though electromagnets are used as the first and second magnetic materials 310 and 320, the first and second magnetic materials 310 and 320 move in position in a state where the attached positions of the first and second electrodes 210 and 220 are fixed, and accordingly, an angle θ between a first line connecting the centers of the first and second electrodes 210 and 220 and a second line connecting the centers of the first and second magnetic materials 310 and 320 is changed. As the crossing angle θ comes close to 90°, in this case, the electric current path between the electrodes 210 and 220 may become deep in depth. As the crossing angle θ is controlled to increase the depth of the electric current path, as mentioned above, the target region to be electrically stimulated may become deep in depth.

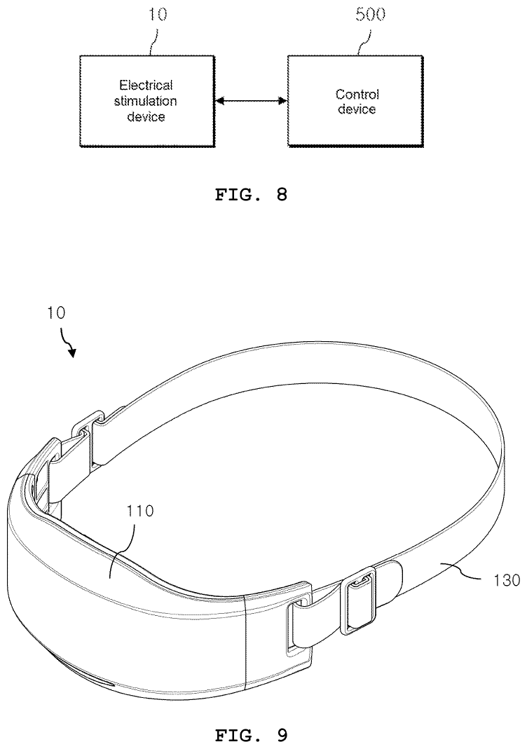

FIGS. 7a to 7c are exemplary views showing methods for controlling the strength B of the magnetic field according to the present invention.

Referring to FIGS. 7a to 7c, the strength B of the magnetic field generated by the first and second magnetic materials 310 and 320 is increasingly controlled in the order of FIG. 7a, FIG. 7b, and FIG. 7c.

For example, if electromagnets are used as the first and second magnetic materials 310 and 320, the size of an electric current supplied to the first and second magnetic materials 310 and 320 is changed, and accordingly, an electric current path between the first and second electrodes 210 and 220 may become controlled in depth.

Even in the case where the size of the electric current supplied to the first and second magnetic materials 310 and 320 is changed to increase the strength B of magnetic field, as mentioned above, the electric current path between the first and second electrodes 210 and 220 may become deep in depth, as shown in FIGS. 6a to 6c.

As the strength B of the magnetic field generated by the first and second magnetic materials 310 and 320 is controlled to increase the depth of the electric current path, the target region to be electrically stimulated may become deep in depth.

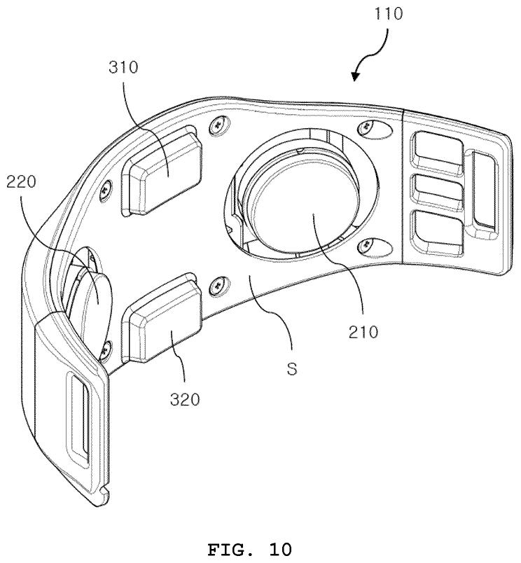

FIG. 8 is a block diagram showing an overall configuration of a noninvasive electrical stimulation system according to the present invention, and the noninvasive electrical stimulation system includes an electrical stimulation device 10 and a control device 500. The repeated explanation of the same parts and operations of the electrical stimulation system as mentioned above with reference to FIGS. 1 to 7c will be avoided herein.

Referring to FIG. 8, a tDCS device is used as the noninvasive electrical stimulation device 10 according to the present invention, and without being limited thereto, various devices, which are capable of applying noninvasive electrical stimulation using electrodes coming into contact with a human body, such as an nVNS device, a tRNS device, and the like, may be used as the noninvasive electrical stimulation device 10.

For example, the electrical stimulation device 10 may be a device configured to allow electrodes to come into contact with a user's forehead region to apply electrical stimulation to his or her brain, and accordingly, an electric current flows to a specific region of his or her head to thus apply electrical stimulation to a target region of the brain.

Hereinafter, for example, an explanation of the electrical stimulation device 10 configured to apply electrical stimulation to the brain will be given, but without being limited thereto, of course, the electrical stimulation device 10 according to the present invention may be applied even in the case where electrical stimulation may be applied to other specific regions except the brain.

The electrical stimulation device 10 includes first and second electrodes for applying electrical stimulation, and the control device 500 generates control signals for controlling operations of the electrical stimulation device 10 and transmits the control signals to the electrical stimulation device 10.

The electrical stimulation device 10 and the control device 500 are connected to each other wiredly or wirelessly to transmit and receive signals and data to and from each other.

In specific, the electrical stimulation device 10 includes an electrode part having first and second electrodes and first and second magnetic materials located on the curved inner surface thereof to allow the first and second magnetic materials to generate a magnetic field in a direction crossing the direction of an electric current between the first and second electrodes. Further, the control device 500 generates a control signal for controlling at least one of a crossing angle θ at which the electric current between the first and second electrodes and the magnetic field generated by the first and second magnetic materials cross each other, and the strength B of the magnetic field generated by the first and second magnetic materials and transmits the control signal to the electrode part of the electrical stimulation device 10.

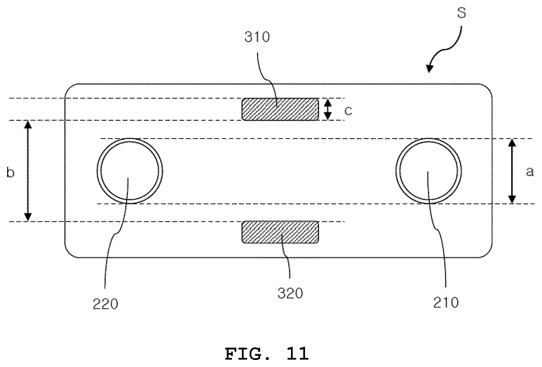

Hereinafter, another noninvasive electrical stimulation device according to the present invention will be explained with referent to FIGS. 9 to 11.

Referring to FIGS. 9 and 10, a noninvasive electrical stimulation device 10 is worn to allow electrodes to come into contact with the forehead of the human body and includes an electrode part 110 and a fixing part 130.

The electrode part 110 includes first and second electrodes 210 and 220 disposed on a curved inner surface S thereof to come into contact with the forehead region of a human body thereof to apply electrical stimulation and first and second magnetic materials 310 and 320 disposed on the curved inner surface S to generate a magnetic filed in a direction crossing the direction of an electric current between the first and second electrodes 210 and 220.

The fixing part 130 has the shape of a band to fix the electrode part 110 to the forehead region, but according to the present invention, the fixing part 130 may not be limited thereto.

The electrical stimulation device 10 may further include a controller for controlling the crossing angle θ at which the electric current between the first and second electrodes 210 and 220 and the magnetic field generated by the first and second magnetic materials 310 and 320 cross each other or the strength B of the magnetic field generated by the first and second magnetic materials 310 and 320.

For example, the controller is disposed inside the electrode part 110 in the form of an integrated circuit (IC) chip with circuits for controlling at least one of the crossing angle θ and the strength B of the magnetic field, and according to the present invention, the controller is not limited thereto.

Further, the control device 500 includes a plurality of buttons for receiving the user's inputs, a display for displaying operating states thereof, and an IC chip disposed therein to generate control signals for controlling the operations of the electrical stimulation device 10.

For example, the control device 500 transmits the control signals to the electrical stimulation device 10 through a cable (not shown) connected to the electrode part 110 of the electrical stimulation device 10, but the electrical stimulation device 10 and the control device 500 transmit and receive the control signals to and from each other through a near filed wireless communication technology such as Bluetooth.

According to the present invention, the control device 500 receives the target region of the brain to be treated or to be electrically stimulated from the user, calculates a depth of an electric current path corresponding to the user's input, and transmits information of a crossing angle θ or the strength B of a magnetic field, as the control signal, to the electrode part 110 of the electrical stimulation device 10 to thus form the calculated depth of the electric current path.

Further, the electrode part 110 receives the control signal from the control device 500, controls the controller disposed therein to move the first and second magnetic materials 310 and 320 in position according to the crossing angle θ contained in the control signals, and allows the electric current between the first and second electrodes 210 and 220 and the magnetic field generated by the first and second magnetic materials 310 and 320 to cross each other at the corresponding crossing angle θ.

Further, the electrode part 110 changes the size of the electric current supplied to the first and second electrodes 210 and 220 through the controller disposed therein to thus allow the strength B of the magnetic field generated by the first and second magnetic materials 310 and 320 to be controlled to have the corresponding strength B.

Referring to FIG. 11, a distance b between the first and second magnetic materials 310 and 320 is greater than a width a of each electrode 210 or 220 so as to maximize an influence of the magnetic field generated by the first and second magnetic materials 310 and 320 on the electric current between the first and second electrodes 210 and 220.

Further, if permanent magnets are used as the first and second electrodes 210 and 220, a width c of each magnetic material 310 or 320 is set according to the strength of a magnetic field as required.

The noninvasive electrical stimulation method according to the present invention may be made in the form of a program to be executed on a computer. Further, the program may be stored in a computer-readable storage medium, and examples of the computer-readable storage medium include read-only memory (ROM), random-access memory (RAM), CD-ROM, magnetic tapes, floppy disks, optical data storage devices, and the like.

The computer-readable storage medium is distributed to a computer system connected thereto through a network, and accordingly, computer-readable codes are stored and executed in a distributed way. Further, functional programs, codes, and code segments for carrying out the method according to the present invention can be easily inferred by programmers in the art.

While the foregoing examples are illustrative of the principle of the present invention in one or more particular applications, it will be apparent to those or ordinary skill in the art that numerous modifications in form, usage, and details of implementation can be made without the exercise of inventive faculty, and without departing from the principles and concepts of the invention. Accordingly, it is not intended that the invention be limited, except as by the claims set forth below.

What is claimed is:

1. A noninvasive electrical stimulation method comprising the steps of:
arranging first and second electrodes for applying electrical stimulation to a human body, and first and second magnetic materials to thus allow the first and second magnetic materials to generate a magnetic field in a direction crossing the direction of an electric current between the first and second electrodes; and
controlling at least one of a crossing angle (θ) at which the electric current between the first and second electrodes and the magnetic field generated by the first and second magnetic materials cross each other and the strength (B) of the magnetic field generated by the first and second magnetic materials.

2. The noninvasive electrical stimulation method according to claim 1, wherein the first and second magnetic materials are permanent magnets or electromagnets with different polarities from each other.

3. The noninvasive electrical stimulation method according to claim 2, wherein if the permanent magnets are used as the first and second magnetic materials, an angle between a first line connecting the centers of the first and second electrodes and a second line connecting the centers of the first and second magnetic materials is controlled.

4. The noninvasive electrical stimulation method according to claim 2, wherein if the electromagnets are used as the first and second magnetic materials, a size of the electric current supplied to the first and second magnetic materials is controlled to generate the magnetic field.

5. The noninvasive electrical stimulation method according to claim 1, wherein as the crossing angle (θ) comes close to 90°, an electric current path between the first and second electrodes becomes deep in depth.

6. The noninvasive electrical stimulation method according to claim 1, wherein as the strength (B) of the magnetic field increases, an electric current path between the first and second electrodes becomes deep in depth.

7. The noninvasive electrical stimulation method according to claim 5, wherein as the electric current path between the first and second electrodes becomes deep in depth, a target region to be electrically stimulated increases in depth.

8. The noninvasive electrical stimulation method according to claim 6, wherein as the electric current path between the first and second electrodes becomes deep in depth, a target region to be electrically stimulated increases in depth.

9. A noninvasive electrical stimulation device comprising:
an electrode part having first and second electrodes adapted to apply electrical stimulation;
a magnetic force generation part having first and second magnetic materials adapted to generate a magnetic field in a direction crossing the direction of an electric current between the first and second electrodes; and
a controller for controlling at least one of a crossing angle (θ) at which the electric current between the first and second electrodes and the magnetic field generated by the first and second magnetic materials cross each other and the strength (B) of the magnetic field generated by the first and second magnetic materials.

10. The noninvasive electrical stimulation device according to claim 9, wherein if permanent magnets with different polarities from each other are used as the first and second magnetic materials, the controller moves the first and second magnetic materials in position to control the crossing angle (θ) between a first line connecting the centers of the first and second electrodes and a second line connecting the centers of the first and second magnetic materials.

11. The noninvasive electrical stimulation device according to claim 9, wherein if electromagnets with different polarities from each other are used as the first and second magnetic materials, the controller controls a size of the electric current supplied to the first and second magnetic materials to generate the magnetic field.

12. The noninvasive electrical stimulation device according to claim 9, wherein as the crossing angle (θ) comes close to 90°, an electric current path between the first and second electrodes becomes deep in depth.

13. The noninvasive electrical stimulation device according to claim 9, wherein as the strength (B) of the magnetic field increases, an electric current path between the first and second electrodes becomes deep in depth.
